(19) United States
(12) Patent Application Publication
(10) Pub. No.: US 2022/0010260 A1
(43) Pub. Date: Jan. 13, 2022

(54) DEVICE OF MEASURING AMOUNT OF LIPID ACCUMULATION IN MICROALGAE AND METHOD OF MEASURING AMOUNT OF LIPID ACCUMULATION IN MICROALGAE

(71) Applicant: AZBIL CORPORATION, Tokyo (JP)

(72) Inventor: Norio HASEGAWA, Chiyoda-ku (JP)

(73) Assignee: AZBIL CORPORATION, Tokyo (JP)

(21) Appl. No.: 17/353,902

(22) Filed: Jun. 22, 2021

(30) Foreign Application Priority Data
Jul. 10, 2020 (JP) 2020-119350

(51) Int. Cl. C12M 1/34; C12M 1/36; G01N 21/64

(52) U.S. Cl. CPC C12M 41/32; G01N 21/6458; G01N 21/6486; C12M 41/48

(57) ABSTRACT

A device of measuring an amount of lipid accumulation in microalgae includes a flow cell through which a fluid containing the microalgae is supplied to flow, an excitation light source irradiating the flow cell with excitation light, a fluorescence detector detecting autofluorescence generated from a chloroplast of each of the microalgae that have been irradiated with the excitation light, a scattered light detector detecting scattered light caused by each of the microalgae that have been irradiated with the excitation light, and an arithmetic unit calculating a size of the microalga from intensity of the scattered light, calculating a fluorescence density corresponding to intensity of the autofluorescence generated from the chloroplast per unit size of the microalga based on both the intensity of the autofluorescence generated from the chloroplast and the size of the microalga, and calculating an amount of lipid accumulation per microalga from the fluorescence density.

### CROSS-REFERENCE TO RELATED APPLICATIONS

The present application is based on and claims priority to Japanese Application No. 2020-119350, filed Jul. 10, 2020, the entire contents of which are incorporated herein by reference.

### BACKGROUND

### 1. Field of the Disclosure

The present disclosure relates to a device of measuring an amount of lipid accumulation in microalgae and a method of measuring an amount of lipid accumulation in microalgae.

### 2. Description of the Related Art

In consideration of that carbon dioxide in the atmosphere can be utilized as resources, attention is focused on using, as biofuel, lipids (such as fatty acid ester) which are produced by and accumulated in microalgae with the aid of photosynthetic potential. The biofuel is produced from the microalgae by culturing the microalgae, ending the culture at appropriate timing, and taking out the lipids from the microalgae or a fluid containing the microalgae. The appropriate timing may be time at which a total yield of the lipids is maximized in an entire culture process.

Knowing a total amount of lipid accumulation in a larger number of microalgae is required in management of the culture process. Furthermore, knowing an amount of lipid accumulation per microalga is important to check culture efficiency and to determine conditions of the culture process. The conditions of the culture process, such as concentrations of culture medium components, temperature of a culture solution, pH of the culture solution, dissolved oxygen in the culture solution, and an amount of carbon dioxide absorbed by the microalgae, are measured and monitored to efficiently culture the microalgae. As an example of known methods of measuring the amount of lipid accumulation in the microalgae, there is proposed a method of sampling a fluid containing the microalgae, extracting lipids from the sampled fluid, and weighing the extracted lipids. As another example of the known methods of measuring the amount of lipid accumulation in the microalgae, there is also proposed a method of staining lipids in the microalgae with a fluorescent dye and observing the microalgae with a fluorescence microscope (see, for example, Kawamura, K et al. “Determining of the optimal cultivation strategy for microalgae for biodiesel production using flow cytometric monitoring and mathematical modeling,” (2018) Biomass and Bioenergy, 117, 24-31). Meanwhile, it is further proposed to quantitate the amount of lipid accumulation in the microalgae from change of a color tone of a suspension containing a large number of microalgae (see, Japanese Unexamined Patent Application Publication No. 2017-3475).

However, the method of extracting the lipids in the microalgae and weighing the extracted lipids cannot process many samples because an extraction operation is intricate and needs a lot of time and efforts. The method of staining the lipids in the microalgae with the fluorescent dye requires pretreatment for the staining and needs a lot of time and efforts. In addition, due care has to be paid in handling of the fluorescent dye from the viewpoint of safety, and treatment of waste including a staining agent is also intricate.

Furthermore, the method of quantitating the amount of lipid accumulation in the microalgae from the change of the color tone of the suspension containing the large number of microalgae can measure the total amount of lipid accumulation in the large number of microalgae but cannot accurately measure the amount of lipid accumulation per microalga. The reason is that Japanese Unexamined Patent Application Publication No. 2017-3475 is difficult to measure an exact cell amount because a G (Green) component strongly correlated with the cell amount reduces as an oil component increases. In addition, it is reported by Wayama, M et al. “Three-dimensional ultrastructural study of oil and astaxanthin accumulation during encystment in the green alga Haema tococcus pluvialis,” (2013) PLOS ONE, 8, e53618 that the lipids produced by and accumulated in the microalgae are resulted from change of cell membranes of cell organelles such as chloroplasts.

### SUMMARY

In view of the above-described situation, one object of the present disclosure is to provide a device of measuring an amount of lipid accumulation in microalgae and a method of measuring an amount of lipid accumulation in microalgae, the device and the method enabling lipids contained in the microalgae to be observed in a simple and quick manner.

An embodiment of the present disclosure provides a device of measuring an amount of lipid accumulation in microalgae, the device including (a) a flow cell through which a fluid containing the microalgae is supplied to flow, (b) an excitation light source irradiating the flow cell with excitation light, (c) a fluorescence detector detecting autofluorescence generated from a chloroplast of each of the microalgae that have been irradiated with the excitation light, (d) a scattered light detector detecting scattered light caused by each of the microalgae that have been irradiated with the excitation light, and (e) an arithmetic unit calculating a size of the microalga from intensity of the scattered light, calculating a fluorescence density corresponding to intensity of the autofluorescence generated from the chloroplast per unit size of the microalga based on both the intensity of the autofluorescence generated from the chloroplast and the size of the microalga, and calculating an amount of lipid accumulation per microalga from the fluorescence density. The autofluorescence generated from the chloroplast of the microalga may be red light.

In the above-described device of measuring the amount of lipid accumulation in the microalgae, the arithmetic unit may calculate an amount of lipid accumulation per number of the microalgae at a certain concentration from the fluorescence density and may calculate the amount of lipid accumulation per microalga from the amount of lipid accumulation per number of the microalgae at the certain concentration.

In the above-described device of measuring the amount of lipid accumulation in the microalgae, the arithmetic unit may further calculate a concentration of the microalgae from a volume of the fluid having passed through the flow cell during a unit time and a number of detection signals for the scattered light caused by the microalgae during the unit time, and may further calculate a concentration of lipids of the microalgae from the amount of lipid accumulation per microalga and the concentration of the microalgae.

In the above-described device of measuring the amount of lipid accumulation in the microalgae, the arithmetic unit may determine that it is time to end culture of the microalgae, when the amount of lipid accumulation per microalga and/or the concentration of the lipids of the microalgae exceeds a predetermined discriminant value.

In the above-described device of measuring the amount of lipid accumulation in the microalgae, an output unit may issue a command to stop the culture in a supply source for the fluid containing the microalgae in accordance with the determination that it is time to end the culture.

In the above-described device of measuring the amount of lipid accumulation in the microalgae, the arithmetic unit may evaluate a state of the microalgae based on the amount of lipid accumulation per microalga and/or the concentration of the lipids of the microalgae and may determine that it is time to adjust culture conditions in a supply source for the fluid containing the microalgae.

In the above-described device of measuring the amount of lipid accumulation in the microalgae, an output unit may issue a command to adjust the culture conditions in the supply source for the fluid containing the microalgae in accordance with the determination that it is time to adjust the culture conditions.

In the above-described device of measuring the amount of lipid accumulation in the microalgae, a storage unit may record the intensity of the detected autofluorescence from the chloroplast and the intensity of the detected scattered light in chronological order. The storage unit may further record the calculated amount of lipid accumulation per microalga and the calculated concentration of the lipids of the microalgae in chronological order.

In the above-described device of measuring the amount of lipid accumulation in the microalgae, a display unit may display the calculated amount of lipid accumulation per microalga and/or the calculated concentration of the lipids of the microalgae.

Another embodiment of the present disclosure provides a method of measuring an amount of lipid accumulation in microalgae, the method including (a) supplying a fluid containing the microalgae to flow through a flow cell, (b) irradiating the flow cell with excitation light, (c) detecting autofluorescence generated from a chloroplast of each of the microalgae that have been irradiated with the excitation light, (d) detecting scattered light caused by each of the microalgae that have been irradiated with the excitation light, (e) calculating a size of the microalga from intensity of the scattered light, (f) calculating a fluorescence density corresponding to intensity of the autofluorescence generated from the chloroplast per unit size of the microalga based on both the intensity of the autofluorescence generated from the chloroplast and the size of the microalga, and (g) calculating an amount of lipid accumulation per microalga from the fluorescence density. The autofluorescence generated from the chloroplast of the microalga may be red light.

The above-described method of measuring the amount of lipid accumulation in the microalgae may further include calculating an amount of lipid accumulation per number of the microalgae at a certain concentration from the fluorescence density, and calculating the amount of lipid accumulation per microalga from the amount of lipid accumulation per number of the microalgae at the certain concentration.

The above-described method of measuring the amount of lipid accumulation in the microalgae may further include calculating a concentration of the microalgae from a volume of the fluid having passed through the flow cell during a unit time and a number of detection signals for the scattered light caused by the microalgae during the unit time, and calculating a concentration of lipids of the microalgae from the amount of lipid accumulation per microalga and the concentration of the microalgae.

The above-described method of measuring the amount of lipid accumulation in the microalgae may include determining that it is time to end culture of the microalgae, when the amount of lipid accumulation per microalga and/or the concentration of the lipids of the microalgae exceeds a predetermined discriminant value.

The above-described method of measuring the amount of lipid accumulation in the microalgae may include issuing a command to stop the culture in a supply source for the fluid containing the microalgae in accordance with the determination that it is time to end the culture.

The above-described method of measuring the amount of lipid accumulation in the microalgae may include evaluating a state of the microalgae based on the amount of lipid accumulation per microalga and/or the concentration of the lipids of the microalgae, and determining that it is time to adjust culture conditions in a supply source for the fluid containing the microalgae.

The above-described method of measuring the amount of lipid accumulation in the microalgae may include issuing a command to adjust the culture conditions in the supply source for the fluid containing the microalgae in accordance with the determination that it is time to adjust the culture conditions.

The above-described method of measuring the amount of lipid accumulation in the microalgae may include recording the intensity of the detected autofluorescence from the chloroplast and the intensity of the detected scattered light in chronological order. The above-described method may further include recording the calculated amount of lipid accumulation per microalga and the calculated concentration of the lipids of the microalgae in chronological order.

The above-described method of measuring the amount of lipid accumulation in the microalgae may include displaying the calculated amount of lipid accumulation per microalga and/or the calculated concentration of the lipids of the microalgae.

A still another embodiment of the present disclosure provides a method of controlling culture of microalgae, the method including (a) supplying a fluid containing the microalgae to flow through a flow cell and irradiating the flow cell with excitation light, (b) detecting autofluorescence generated from a chloroplast of each of the microalgae that have been irradiated with the excitation light, (c) detecting scattered light caused by each of the microalgae that have been irradiated with the excitation light, (d) calculating a size of the microalga from intensity of the scattered light, (e) calculating a concentration of the microalgae from a volume of the fluid having passed through the flow cell during a unit time and a number of detection signals for the scattered light caused by the microalga during the unit time, (f) calculating a fluorescence density corresponding to intensity of the autofluorescence generated from the chloroplast per unit size of the microalga based on both the intensity of the autofluorescence generated from the chloroplast and the size of the microalga, (g) calculating an amount of lipid accumulation per number of the microalgae at a certain concentration from the fluorescence density, (h) calculating the amount of lipid accumulation per microalga from the amount of lipid accumulation per number of the microalgae at the certain concentration, (i) calculating a concentration of lipids of the microalgae from the amount of lipid accumulation per microalga and the concentration of the microalgae, (j) determining that it is time to end culture of the microalgae, when the amount of lipid accumulation per microalga and/or the concentration of the lipids of the microalgae exceeds a predetermined discriminant value, and (k) ending the culture of the microalgae in accordance with the determination that it is time to end the culture. The autofluorescence generated from the chloroplast of the microalga may be red light.

The above-described method of controlling the culture of the microalgae may further include evaluating a state of the microalgae based on the amount of lipid accumulation per microalga and/or the concentration of the lipids of the microalgae and determining that it is time to adjust culture conditions in a supply source for the fluid containing the microalgae, and adjusting the culture conditions in the supply source for the fluid containing the microalgae in accordance with the determination that it is time to adjust the culture conditions.

The above-described method of controlling the culture of the microalgae may include recording the intensity of the detected autofluorescence from the chloroplast and the intensity of the detected scattered light in chronological order. The above-described method may further include recording the calculated amount of lipid accumulation per microalga and the calculated concentration of the lipids of the microalgae in chronological order.

The above-described method of controlling the culture of the microalgae may include displaying the calculated amount of lipid accumulation per microalga and/or the calculated concentration of the lipids of the microalgae.

According to the present disclosure, the device of measuring the amount of lipid accumulation in the microalgae and the method of measuring the amount of lipid accumulation in the microalgae can be obtained each of which enables the lipids contained in the microalgae to be observed in a simple and quick manner.

### BRIEF DESCRIPTION OF THE DRAWINGS

FIG. 1 is a block diagram of a device of measuring an amount of lipid accumulation in microalgae according to an embodiment;

FIG. 2 is a flowchart of a method of controlling culture of the microalgae according to an embodiment;

FIG. 3 is a graph representing a relationship of a fluorescence density of a chloroplast to the amount of lipid accumulation per number of the microalgae at a certain concentration in the embodiment; and

FIG. 4 illustrates the progress of the culture of the microalgae in patterns (A) and (B).

### DETAILED DESCRIPTION

Embodiments of the present disclosure will be described below. However, the description and the drawings forming part of the present disclosure should not be considered as limiting the present disclosure. It should be understood that various alternative techniques and application techniques will be apparent to those skilled in the art from the present disclosure, and that the present disclosure includes other various embodiments not described here.

The embodiments of the present disclosure will be described in detail below with reference to the drawings.

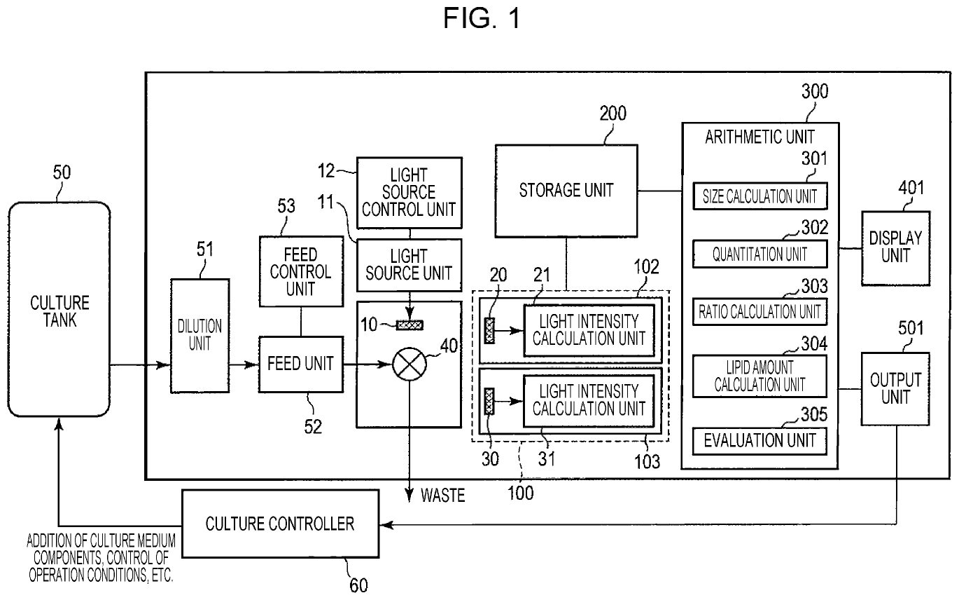

A device of measuring an amount of lipid accumulation in microalgae according to the embodiment includes, as illustrated in FIG. 1, a flow cell 40 through which a fluid containing the microalgae is supplied to flow, an excitation light source 10 irradiating the flow cell 40 with excitation light, a fluorescence detector 102 detecting autofluorescence generated from lipid (chloroplast) in each of the microalgae that have been irradiated with the excitation light, a scattered light detector 103 detecting scattered light caused by the microalga, and an arithmetic unit 300 calculating a size of the microalga from intensity of the scattered light, calculating a fluorescence density corresponding to intensity of the autofluorescence from the chloroplast per unit size of the microalga based on both the intensity of the autofluorescence from the chloroplast and the size of the microalga, and calculating an amount of lipid accumulation per microalga from the fluorescence density. The arithmetic unit 300 is constituted by, for example, a central processing unit (CPU), and so on. The lipid contained in the microalga is also called an oil body.

The excitation light source 10 irradiates the fluid flowing through the flow cell 40 with the excitation light in a wide wavelength range. For example, a light emitting diode (LED) or a laser can be used as the excitation light source 10. The excitation light is, for example, blue light at a wavelength of 450 nm to 495 nm. However, the wavelength and the color of the excitation light are not limited to the above-mentioned examples. The excitation light may be visible light, such as violet light, other than the blue light, or may be ultraviolet light. The wavelength band of the excitation light may be set with a filter such as a bandpass filter. The excitation light forms a focus inside the flow cell 40. A light source unit 11 for supplying electric power to the excitation light source 10 is connected to the excitation light source 10. A light source control unit 12 for controlling the electric power supplied to the light source unit 11 and controlling the excitation light source 10 is connected to the light source unit 11.

The flow cell 40 is transparent to the excitation light and is made of, for example, quartz. The flow cell 40 has such an inner diameter as allowing the microalgae to flow therethrough almost one by one. The flow cell 40 is in the form of a round tube or a square tube. The fluid flowing through the flow cell 40 intersects the excitation light.

The microalgae are algae existing as unicellular organisms with a size of, for example, several micrometers to several ten micrometers. The microalgae are also called phytoplanktons in some cases. Furthermore, the microalgae produce hydrocarbons, for example. Examples of the microalgae may be Botryococcus braunii, Aurantiochytrium, Pseudochoricystis ellipsoidea, Scenedesmus (Desmodesmus), Chlorella, Dunaliella, Arthrospira (Spirulina), Euglena, Nannochloropsis, Haematococcus, and Microcystis aeruginosa.

The microalgae supplied to flow through the flow cell 40 are not stained with a fluorescent dye in advance. The flow cell 40 is coupled, through piping, to a culture tank 50 in which the microalgae are cultured, thus allowing the microalgae to be sent to the flow cell 40 from the culture tank 50 over time. The culture tank 50 is a supply source of the fluid containing the microalgae flowing through the flow cell. The microalgae having flowed through the flow cell 40 is wasted, for example, to the outside of the device through piping. Instead, the microalgae having flowed through the flow cell 40 may be returned to the culture tank 50, described later, through piping.

The flow cell 40 is connected, through a dilution unit 51 and a feed unit 52, for example, to the culture tank 50 in which the microalgae are cultured. The fluid containing the microalgae being cultured in the culture tank 50 may be supplied, for example, routinely to flow through the flow cell 40. As an alternative, the fluid containing the microalgae being cultured in the culture tank 50 may be sampled little by little and then supplied to flow through the flow cell 40. The dilution unit 51 in which the fluid is diluted is connected to the culture tank 50. The dilution unit 51 adds water to the fluid supplied from the culture tank 50 and adjusts a concentration of the microalgae in the fluid flowing through the flow cell 40. The feed unit 52 for feeding the diluted fluid to the flow cell 40 at a constant flow rate is connected to the dilution unit 51. A feed control unit 53 for controlling the flow rate of the fluid fed to the flow cell 40 by the feed unit 52 is connected to the feed unit 52.

When any microalga is contained in the fluid flowing through the flow cell 40, a chloroplast of the microalga irradiated with the excitation light generates autofluorescence that is red light at a wavelength of about 650 nm to 730 nm. A wavelength peak of the autofluorescence generated from the chloroplast exists in a range of about 680 nm to 700 nm. Intensity of the autofluorescence generated from the chloroplast reflects a size of the chloroplast contained in the microalga. Furthermore, the microalga irradiated with the excitation light generates scattered light due to Mie scattering. Intensity of the scattered light reflects a size of the whole of one microalga. Here, the term “size” implies, for example, a diameter, an area, or a volume. For example, when each microalga and each chloroplast have a shape that can be approximated by a particle, the “size” may be a particle diameter.

As illustrated in FIG. 1, a light receiving unit 100 includes a fluorescence detector 102 and a scattered light detector 103. The fluorescence detector 102 detecting the autofluorescence generated from the chloroplast of the microalga includes a light receiving element 20 that receives the autofluorescence generated from the chloroplast of the microalga. A filter for setting a wavelength range of the light receivable by the light receiving element 20, such as an absorption filter, may be disposed in front of the light receiving element 20. For example, a solid-state image sensing device such as a CCD (charge coupled device) image sensor, a photosensor of internal photoelectric effect (photovoltaic effect) type, such as a photodiode, or a photosensor of external photoelectric effect type, such as a photomultiplier, can be used as the light receiving element 20. Upon receiving the autofluorescence generated from the chloroplast, the light receiving element 20 converts light energy to electrical energy. An amplifier for amplifying a current generated by the light receiving element 20 may be connected to the light receiving element 20. An amplifier power supply for supplying electric power to the amplifier may be connected to the amplifier.

Furthermore, a light intensity calculation unit 21 for calculating the intensity of the autofluorescence, which has been generated from the chloroplast and received by the light receiving element 20, based on a magnitude of an electrical signal generated by the light receiving element 20 is connected to the light receiving element 20. The light intensity calculation unit 21 calculates the intensity of the autofluorescence generated from the chloroplast based on, for example, an area of a spectrum of the detected autofluorescence. The light intensity calculation unit 21 may calculate the intensity of the autofluorescence generated from the chloroplast by using image analysis software. Instead, the light intensity calculation unit 21 may calculate the intensity of the autofluorescence generated from the chloroplast based on a magnitude of the current amplified by the amplifier. A storage unit 200 is connected to the light intensity calculation unit 21. The intensity of the autofluorescence generated from the chloroplast, after having been calculated by light intensity calculation unit 21, is stored in the storage unit 200.

The scattered light detector 103 includes a scattered light receiving element 30 that receives the scattered light. For example, a solid-state image sensing device such as a CCD (charge coupled device) image sensor, a photosensor of internal photoelectric effect (photovoltaic effect) type, such as a photodiode, or a photosensor of external photoelectric effect type, such as a photomultiplier, can be used as the scattered light receiving element 30. Upon receiving light, the scattered light receiving element 30 converts light energy to electrical energy. An amplifier for amplifying a current generated by the scattered light receiving element 30 may be connected to the scattered light receiving element 30. An amplifier power supply for supplying electric power to the amplifier may be connected to the amplifier.

Moreover, a light intensity calculation unit 31 for calculating the intensity of the scattered light, which has been received by the scattered light receiving element 30, based on a magnitude of an electrical signal generated by the scattered light receiving element 30 is connected to the scattered light receiving element 30. The light intensity calculation unit 31 calculates the intensity of the scattered light based on, for example, an area of a spectrum of the scattered light having been detected. The light intensity calculation unit 31 may calculate the intensity of the scattered light by using image analysis software. Instead, the light intensity calculation unit 31 may calculate the intensity of the scattered light based on a magnitude of the current amplified by the amplifier. The storage unit 200 is connected to the light intensity calculation unit 31. The intensity of the scattered light, after having been calculated by light intensity calculation unit 31, is stored in the storage unit 200.

When the fluid flows through the flow cell 40, the excitation light source 10 emits the excitation light, fluorescence detector 102 measures the intensity of the autofluorescence generated from the chloroplast of the microalga, and the measured intensity is stored in the storage unit 200. Furthermore, the scattered light detector 103 measures the scattered light caused by the microalga, and the measured intensity of the scattered light is stored in the storage unit 200. The autofluorescence and the scattered light that have been detected at the same time can be regarded as being derived from the same one individual of the microalga. Moreover, when the scattered light and the autofluorescence generated from the chloroplast have been detected at the same time, such a phenomenon can be regarded as indicating that one microalga has intersected the excitation light. Accordingly, the number of the microalgae having passed through the flow cell 40 can be counted from the number of times that the scattered light and the autofluorescence generated from the chloroplast have been detected at the same time.

The storage unit 200 stores the intensity of the autofluorescence generated from the chloroplast of the microalga and the intensity of the scattered light caused by the microalga. The storage unit 200 may further add time information, such as date and time of the detection, to information regarding the intensity of the scattered light caused by one microalga and the intensity of the autofluorescence generated from the chloroplast of the microalga, and then store the combined information in chronological order.

For example, by repeatedly measuring the intensity of the scattered light caused by the microalga and the intensity of the autofluorescence generated from the chloroplast of the microalga for a certain period, the information regarding the intensity of the scattered light caused by the microalga and the intensity of the autofluorescence generated from the chloroplast of the microalga is accumulated in the storage unit 200. Thus, change over time of the intensity of the scattered light caused by the microalga, change over time of the intensity of the autofluorescence generated from the lipid of the microalga, and change over time of the intensity of the autofluorescence generated from the chloroplast of the microalga are recorded.

The arithmetic unit 300 is connected to the storage unit 200. The arithmetic unit 300 includes a size calculation unit 301. The size calculation unit 301 calculates the size of the microalga based on the intensity of the scattered light caused by the microalga. The size calculation unit 301 may calculate the size of the microalga based on a previously obtained relationship between the intensity of the scattered light and the size of the microalga. The storage unit 200 may record change over time of the size of the microalga which has been calculated by the size calculation unit 301.

The arithmetic unit 300 includes a quantitation unit 302. The quantitation unit 302 calculates a concentration of the microalgae from a volume of the fluid having passed through the flow cell 40 during a unit time and the number of detection signals for the scattered light caused by the microalgae during the unit time. The quantitation unit 302 may further calculate an amount of the microalgae from the volume of the fluid having passed through the flow cell 40 during the unit time, the number of the detection signals for the scattered light caused by the microalgae during the unit time, and the intensity of the scattered light caused by the microalgae during the unit time. For example, assuming that a horizontal axis denotes the number of the detection signals for the scattered light caused by the microalgae during the unit time and a vertical axis denotes intensity of each detection signal, the quantitation unit 302 calculates, as the amount of the microalgae, an integral value of a relation formula between the intensity of each detection signal and the number of the detection signals.

Moreover, the quantitation unit 302 calculates the concentration of the microalgae per unit fluid volume by dividing the amount of the microalgae by the volume of the fluid having passed through the flow cell 40 during the unit time. For example, the quantitation unit 302 calculates the concentration of the microalgae by dividing the number of the signals each generated upon detection of the scattered light from the microalga during the unit time by the volume of the fluid having passed through the flow cell 40 during the unit time. The storage unit 200 may record changes over time of the amount and the concentration of the microalgae which have been calculated by the quantitation unit 302.

The arithmetic unit 300 includes a ratio calculation unit 303. The ratio calculation unit 303 calculates the fluorescence density corresponding to the intensity of the autofluorescence from the chloroplast per unit size of the microalga based on both the intensity of the autofluorescence from the chloroplast of the microalga and the size of the microalga. For example, the ratio calculation unit 303 reads out, from the storage unit 200, the intensity of the autofluorescence generated from the chloroplast of each microalga. The ratio calculation unit 303 further reads out, from the storage unit 200, the size of the microalga. Moreover, the ratio calculation unit 303 divides the intensity of the autofluorescence from the chloroplast by the size of the microalga, thereby calculating the intensity of the autofluorescence from the chloroplast per unit size of the microalga (namely, the fluorescence density). The ratio calculation unit 303 may read out, from the storage unit 200, the intensity of the scattered light caused by the microalga instead of the size of the microalga and may calculate the fluorescence density from a read-out value. As described above, the intensity of the autofluorescence generated from the chloroplast reflects the size of the chloroplast of the microalga. Therefore, the fluorescence density reflects the size of the chloroplast per unit volume of the whole of each microalga. The storage unit 200 may record change over time of the fluorescence density calculated by the ratio calculation unit 303.

The arithmetic unit 300 includes a lipid amount calculation unit 304. The lipid amount calculation unit 304 calculates the amount of lipid accumulation per (one) microalga (hereinafter also called a “unit yield”) from the fluorescence density.

For example, the storage unit 200 stores a previously obtained relation formula between the intensity of the autofluorescence generated from the chloroplast per unit size of the microalga (namely, the fluorescence density) and the amount of lipid accumulation per number of the microalgae at a certain concentration. An example of the relation formula will be described in detail later. For example, the lipid amount calculation unit 304 reads out the fluorescence density of each microalga from the storage unit 200. The lipid amount calculation unit 304 further reads out the above-mentioned relation formula from the storage unit 200. For example, the lipid amount calculation unit 304 calculates, based on the above-mentioned relation formula, the amount of lipid accumulation per number of the microalgae at the certain concentration from the fluorescence density calculated by the ratio calculation unit 303. Furthermore, the lipid amount calculation unit 304 divides the calculated amount of lipid accumulation per number of the microalgae at the certain concentration by the relevant certain concentration, thereby calculating the amount of lipid accumulation per (one) microalga (namely, the unit yield).

Moreover, the lipid amount calculation unit 304 may calculate a concentration of the lipids of the microalgae (hereinafter also called a “total yield”) from the calculated amount of lipid accumulation per (one) microalga and the concentration of the microalgae. The concentration of the lipids of the microalgae is given as the amount of lipid accumulation per unit volume of the fluid containing the microalgae. For example, the lipid amount calculation unit 304 may read out the concentration of the microalgae, having been calculated by the quantitation unit 302, from the storage unit 200. Then, the lipid amount calculation unit 304 may multiply the calculated amount of lipid accumulation per (one) microalga (namely, the unit yield) by the concentration of the microalgae, thereby calculating the concentration of the lipids of the microalgae.

The storage unit 200 may store changes over time of the total yield and the unit yield both having been calculated by the lipid amount calculation unit 304. In addition, the storage unit 200 may store change over time of the amount of lipid accumulation per number of the microalgae at the certain concentration, which has been calculated by the lipid amount calculation unit 304.

The above-described relation formula between the intensity (Y) of the autofluorescence generated from the chloroplast per unit size of the microalga (namely, the fluorescence density) and the amount (X) of lipid accumulation per number of the microalgae at the certain concentration is expressed by, for example, the following exponential function formula (1).

Y=aX^{-b} (1)

Y: intensity of the autofluorescence generated from the chloroplast per unit size of the microalga (fluorescence density)

X: amount of lipid accumulation per number of the microalgae at the certain concentration (equivalent to fluorescence intensity due to fluorescence agents in the lipids)

a, b: coefficients determined by experiments

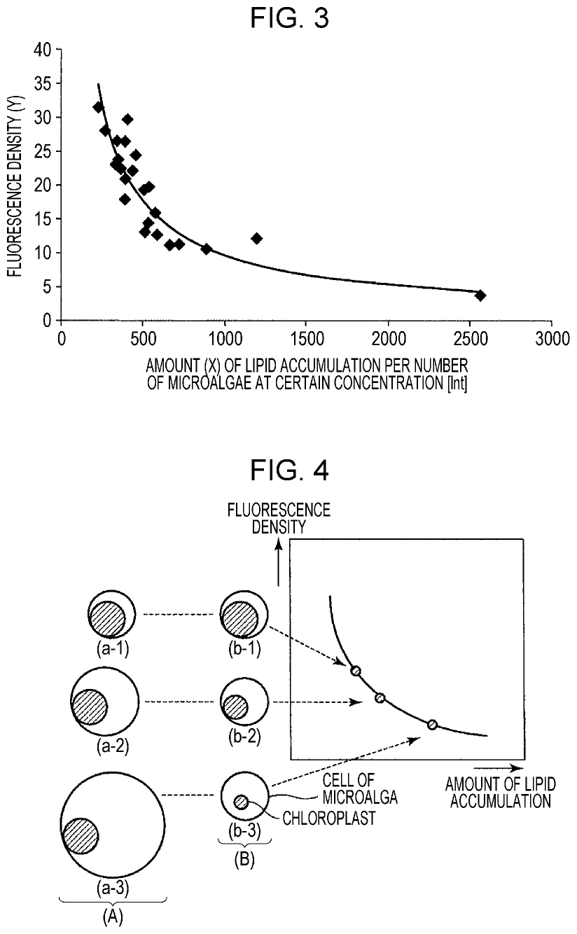

In an example, the formula (1) is represented by a graph depicted in FIG. 3 with a=4179.5 and b=0.879 under conditions of Experimental Example described later.

Generally, in an initial stage of culture of microalgae, cell division is active, the size of lipid occupying each microalga is small, and the size of a chloroplast is large. However, when frequency of the cell division reduces with the lapse of culture time, production of the lipid within the microalga progresses and the lipid is accumulated in the microalga. Because the lipid of the microalga is produced from a membrane of a cell organelle such as a chloroplast, the amount of the chloroplast reduces with accumulation of the lipid in the microalga. Thus, the sizes of the lipid and the chloroplast relative to the size of the microalga changes depending on the state of the microalga.

Focusing on the above-described relationship, the inventors have found that the amount of lipid accumulation per microalga (the unit yield) can be calculated from the intensity of the autofluorescence generated from the chloroplast per unit size of the microalga (the fluorescence density). As seen from the graph of FIG. 3, as the fluorescence density (Y) is larger, the amount (X) of lipid accumulation per number of the microalgae at the certain concentration is smaller. On the other hand, it is also seen that as the fluorescence density (Y) is smaller, the amount (X) of lipid accumulation per number of the microalgae at the certain concentration is larger. The amount of lipid accumulation per number of the microalgae at the certain concentration and the amount of lipid accumulation per microalga are in a proportional relationship.

For example, preferably, the lipid amount calculation unit 304 previously determines whether calculating, based on the above-described relation formula, the amount of lipid accumulation per number of the microalgae at the certain concentration from the fluorescence density is appropriate.

For example, the lipid amount calculation unit 304 reads out the change over time of a cell size from the storage unit 200. The lipid amount calculation unit 304 further reads out the change over time of the fluorescence density from the storage unit 200. Then, the lipid amount calculation unit 304 calculates an increase rate (ΔD) of the cell size from the change over time of the cell size. The lipid amount calculation unit 304 further calculates a decrease rate (ΔF_{L}) of the fluorescence density from the change over time of the fluorescence density. Then, the lipid amount calculation unit 304 calculates a determination aid value (K) by applying, to the following equation, not only calculated values of ΔD and ΔF_{L}, but also an increase rate (ΔD_{O}) of the cell size and a decrease rate (ΔF_{LO}) of the fluorescence density, both the rates having been previously measured when creating a calibration curve.

K=(ΔD−ΔD_{O})/(ΔF_{L}−ΔF_{LO}) (2)

An appropriate range of the determination aid value (K) may be stored in the storage unit 200. The appropriate range of the determination aid value (K) is, for example, K≥0. This is based on the thought that, in the above formula, K takes a negative value when the culture process turns to cell hypertrophy instead of lipid production. Alternatively, by using an average value and a standard deviation (σ) of values K that have been previously obtained by repeating experiments, the appropriate range of the determination aid value (K) may be given as a range of the average value±3σ. This is because the culture process is in an objective growing mode when the value K is within the above range. For example, when the value K is within the above range, it can be determined that calculating the amount of lipid accumulation per number of the microalgae at the certain concentration from the fluorescence density by using the above-described relation formula is appropriate. On the other hand, when the value K is outside the above range, it can be determined that calculating the amount of lipid accumulation per number of the microalgae at the certain concentration from the fluorescence density by using the above-described relation formula is inappropriate.

As described above, because the lipid of the microalga is produced from the membrane of the cell organelle such as the chloroplast, the amount of the chloroplast reduces with accumulation of the lipid in the microalga. The above-described relation formula is intended to calculate the amount of lipid accumulation per number of the microalgae at the certain concentration from the fluorescence density by utilizing the above-described relationship. However, when nutrient in the microalga is mainly used to enlarge a cell of the microalga without being used to accumulate the lipid in the microalga, calculating the amount of lipid accumulation from the fluorescence density by using the above-described relation formula is inappropriate in some cases.

FIG. 4 is an explanatory view illustrating two patterns (A) and (B) in progress of the culture of the microalgae. The pattern (B) represents ideal progress of the culture of the microalgae. The pattern (B) corresponds to, for example, the progress of the culture of the microalgae when the relation formula represented by the graph of FIG. 3 is created. The pattern (A) represents the progress of the culture of the microalgae in which the cell is enlarged in comparison with the pattern (B) with the progress of the culture of the microalgae. In the pattern (A), the culture progresses in order of (a-1), (a-2), and (a-3). In the pattern (B), the culture progresses in order of (b-1), (b-2), and (b-3). Here, the fluorescence density (the intensity of the autofluorescence generated from the chloroplast per unit size of the microalga) is the same between (a-1) and (b-1), between (a-2) and (b-2), and between (a-3) and (b-3). However, it is estimated that, in the pattern (A), because the cell of the microalga is enlarged with the progress of the culture, the nutrient in the cell is consumed for the cell hypertrophy and the amount of lipid accumulation is smaller than in the pattern (B). Accordingly, the progress of the culture in the pattern (B) is more preferable than in the pattern (A).

Thus, even when the fluorescence density has the same value, different states of the cell can be known by considering the change over time of the cell size as well. The lipid amount calculation unit 304 can exactly calculate the amount of lipid accumulation by applying the above-described relation formula after previously determining whether the determination aid value K is within the appropriate range.

The above-described relation formula is previously obtained in accordance with the following procedures.

First, microalgae of the same species and the same strain as the microalgae to be measured are prepared, and preculture and main culture are performed on the prepared microalgae in accordance with an ordinary method. Conditions of the preculture and the main culture are set to be, for example, the same as the culture conditions for the microalgae to be measured. The main culture is continued for a certain period (about 1 to 2 weeks) and, after the lapse of one day from the end of the main culture, a culture solution is sampled per one or two days. The sampled culture solution is readjusted to prepare a suspension containing the microalgae after the main culture at a certain concentration (for example, OD₆₈₀=10). At that time, the microalgae after the main culture are centrifuged to remove a culture medium in a supernatant, thus separating cell pellets. Then, the separated cell pellets are suspended into a phosphate-buffered physiological saline solution for adjustment of the concentration. The suspension after the adjustment of the concentration is divided into two. The following measurements are performed on the two suspensions resulting from the division.

One of the two suspensions after the adjustment of the concentration is stained with a fluorescence reagent solution containing a lipid-labeling fluorescence dye. The fluorescence reagent solution is, for example, an ethanol solution containing 1 mg/mL of BODIPY (registered trademark) 493/503 that is an example of the lipid-labeling fluorescence dye. The fluorescence reagent solution is added at a concentration of 0.2%, for example. Then, the fluorescence-stained suspension is analyzed with a fluorescence spectrophotometer to measure intensity of fluorescence generated from fluorescence agents in lipids per number of the microalgae at the certain concentration (for example, OD₆₈₀=10). For example, a laser beam at a wavelength of 493 nm is used as the excitation light. The intensity of the fluorescence generated from the fluorescence agents in the lipids per number of the microalgae at the certain concentration reflects the amount of lipid accumulation per number of the microalgae at the certain concentration. The amount of lipid accumulation per number of the microalgae at the certain concentration can be calculated based on the previously obtained relationship between the intensity of the fluorescence generated from the fluorescence agents in the lipids and the amount of lipid accumulation per number of the microalgae at the certain concentration.

For the other suspension after the concentration adjustment, intensity of scattered light caused by each microalga and intensity of autofluorescence generated from a chloroplast are measured by a microbe analyzer (for example, IMD-W (registered trademark) made by Azbil Corporation which is based on the principle of flowcytometry. Then, the intensity of the autofluorescence generated from the chloroplast per unit size of the microalga (the fluorescence density) is calculated from the measured intensity of the scattered light caused by the microalga and the measured intensity of the autofluorescence generated from the chloroplast. At that time, preferably, the determination aid value K is calculated from the increase rate (ΔD) of the cell size and the decrease rate (ΔF_{L}) of the fluorescence density, and an average and a standard deviation (σ) of the calculated determination aid values K are stored.

Then, results of the above-described measurements are plotted on a graph with a horizontal axis representing the amount (X) of lipid accumulation per number of the microalgae at the certain concentration (equivalent to fluorescence intensity due to the fluorescence agents in the lipids) and a vertical axis representing the fluorescence density (Y). Curve fitting is applied to the plotted points by using an approximate formula expressed by the above-described formula (1). Consequently, the coefficients a and b in the above-described formula (1) are determined. The amount of lipid accumulation per number of the microalgae at the certain concentration is calculated from the fluorescence density by using the above-described formula (1) with both the coefficients thus determined.

The arithmetic unit 300 may further include an evaluation unit 305. The evaluation unit 305 evaluates the state of the microalgae based on the amount of lipid accumulation per microalga (the unit yield) and/or the concentration of the lipids of the microalgae (the total yield).

For example, when the unit yield and/or the total yield exceeds a predetermined discriminant value (for example, an expected unit yield or an expected total yield), the evaluation unit 305 determines that it is time to end the culture of the microalgae. Instead, the evaluation unit 305 may evaluate that the microalgae are in the state suitable to extract the lipids and that it is time to extract the lipids from the microalgae. The predetermined discriminant values for the unit yield and the total yield may be set as appropriate depending on the species of the microalgae, the culture conditions, the use of the extracted lipids, and so on. Preferably, after the unit yield and the total yield have exceeded the predetermined discriminant values, the microalgae are recovered from the culture tank and the lipids are extracted from the microalgae.

Furthermore, the evaluation unit 305 may evaluate the state of the microalgae based on the unit yield and/or the total yield and may determine that it is time to adjust the culture conditions in the supply source of the fluid containing the microalgae. For example, when the unit yield and the total yield do not satisfy the predetermined discriminant values (for example, the expected unit yield and the expected total yield), the evaluation unit 305 may determine that it is time to adjust the culture conditions in the supply source of the fluid containing the microalgae. For example, the evaluation unit 305 may read out the changes over time of the unit yield and the total yield, and if the increase rates of the unit yield and the total yield are small, the evaluation unit 305 may determine that it is time to adjust the culture conditions in the supply source of the fluid containing the microalgae. The supply source of the fluid containing the microalgae is, for example, the culture tank 50. The adjustment of the culture conditions is preferably performed in a manner of, for example, increasing the culture efficiency in the culture tank 50 and optimizing the culture conditions. For example, when the unit yield and the total yield do not satisfy the predetermined discriminant value, the evaluation unit 305 may determine that it is time to make adjustment so as to optimize the culture conditions in the supply source for the fluid containing the microalgae.

Moreover, in accordance with the determination that calculating the amount of lipid accumulation per number of the microalgae at the certain concentration in the lipid amount calculation unit 304 with the above-described relation formula is inappropriate, the evaluation unit 305 may evaluate the state of the microalgae and may determine that it is time to adjust the culture conditions in the supply source of the fluid containing the microalgae. The adjustment of the culture conditions is preferably performed in a manner of, for example, increasing the culture efficiency in the culture tank 50 and optimizing the culture conditions. For example, the adjustment of the culture conditions is performed by adding the culture medium components to the culture tank 50 such that the nutrient is less likely to be used for the cell hypertrophy and is more likely to be used for the lipid accumulation.

The culture conditions in the supply source of the above-mentioned fluid are concentrations of the culture medium components of the culture solution in the culture tank, a concentration of dissolved oxygen in the culture solution, operation conditions (such as a temperature condition, a light condition, and an aeration condition), and so on. The predetermined discriminant values for the unit yield and the total yield may be set as appropriate depending on the species of the microalgae, the culture conditions, the use of the extracted lipids, and so on. The discriminant values for the expected unit yield and the expected total yield, which are used to make the determination that it is time to end the culture and the determination that it is time to adjust the culture conditions in the supply source of the fluid containing the microalgae, may be the same, or the discriminant values used to make the latter determination may be smaller than those used to make the former determination.

A display unit 401 is connected to the arithmetic unit 300. The display unit 401 displays, for example, the changes over time of the unit yield and the total yield, those changes being stored in the storage unit 200. The display unit 401 further displays the changes over time of the intensity of the scattered light caused by the microalga and the intensity of the autofluorescence generated from the chloroplast of the microalga, those changes being stored in the storage unit 200. The display unit 401 still further displays the change over time of the size of the microalga, that change being stored in the storage unit 200.

In addition, the display unit 401 may display the determination result of the evaluation unit 305. For example, when the evaluation unit 305 determines that the unit yield and/or the total yield has reached a target value, the display unit 401 may issue, for example, a message, a sound, or a signal indicating that the unit yield and/or the total yield has reached the target value. For example, a display, a speaker, or a printer may be used as the display unit 401.

The arithmetic unit 300 may be connected to an output unit 501 for outputting the calculation results of the size calculation unit 301, the quantitation unit 302, the ratio calculation unit 303, the lipid amount calculation unit 304, and the evaluation unit 305 to a culture controller 60 that controls the culture conditions in the supply source of the fluid containing the microalgae (namely, the culture tank 50) which is connected to the flow cell 40.

For example, in accordance with the determination result of the evaluation unit 305, namely the determination that it is time to end the culture of the microalgae, the output unit 501 issues, to the culture controller 60, a command to stop the culture in the culture tank 50. As another example, in accordance with the determination result of the evaluation unit 305, namely the determination that it is time to adjust the culture conditions, the output unit 501 issues, to the culture controller 60, a command to adjust the culture conditions in the culture tank 50.

For example, in accordance with the command issued from the output unit 501 to stop the culture of the microalgae in the culture tank 50, the culture controller 60 stops the culture of the microalgae in the culture tank 50. Furthermore, in accordance with the command issued from the output unit 501 to adjust the culture conditions, the culture controller 60 adjusts the culture conditions in the culture tank 50. The adjustment of the culture conditions is performed, for example, by adding the culture medium components to adjust the concentrations of the culture medium components, or by changing the operation conditions such as the temperature condition, the light condition, the aeration condition, and the culture time. The culture conditions are adjusted in a manner of, for example, increasing the culture efficiency and optimizing the culture conditions.

The device of measuring the amount of lipid accumulation in the microalgae may not need to include the output unit 501. A user of the device may manually operate the culture controller 60 to stop the culture or to adjust the culture conditions in accordance with the calculation result or the determination result that is displayed on the display unit 401.

With the above-described device of measuring the amount of lipid accumulation in the microalgae according to this embodiment, by detecting the autofluorescence generated from the chloroplast contained in each microalga, the amount of lipid accumulated and contained in the microalga can be measured without performing fluorescence staining in advance. For example, in the case of culturing a large number of microalgae, the fluorescence staining is not easy to perform on all the microalgae. However, with the device of measuring the amount of lipid accumulation in the microalgae according to this embodiment, the amount of lipids accumulated and contained in the microalgae can be measured over time by continuously supplying the microalgae to flow through the flow cell. Furthermore, since both the unit yield and the total yield can be accurately measured with the device of measuring the amount of lipid accumulation in the microalgae, it is possible not only to know the time to end the culture, but also to check the culture efficiency and to optimize the culture conditions.

Although a method of measuring the amount of lipid accumulation by detecting autofluorescence generated from the lipid of each microalga is also conceivable, measuring the autofluorescence generated from the lipid of the microalga has a problem that the autofluorescence is generated from some species of lipids, but not generated from the other species of lipids. With the device of measuring the amount of lipid accumulation in the microalgae according to this embodiment, since the autofluorescence generated from the chloroplast is measured and the amount of lipid accumulation is calculated based on the measurement value, the amount of lipid accumulation can be easily and accurately determined regardless of the species of the lipids.

Moreover, with the device of measuring the amount of lipid accumulation in the microalgae according to this embodiment, the change over time of the amount of lipid accumulation per microalga and the change over time of the concentration of the lipids of the microalgae can be calculated by measuring the change over time of the autofluorescence generated from each chloroplast. Hence the culture of the microalgae can be further controlled based on the calculation results.

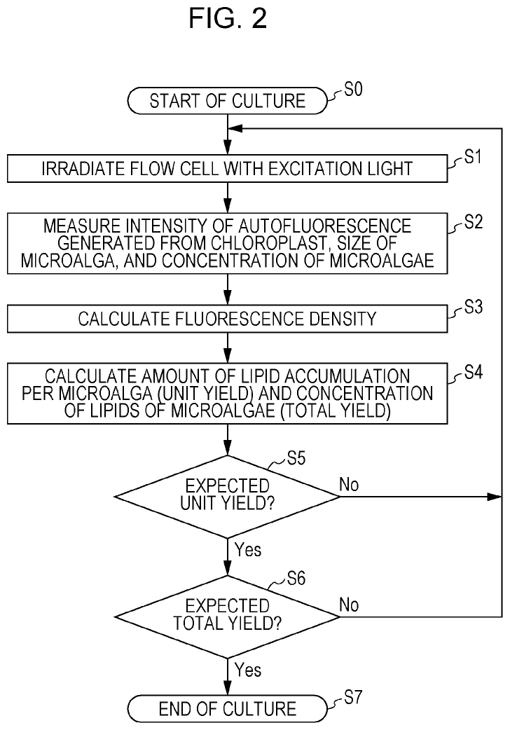

FIG. 2 is a flowchart of a method of controlling the culture of the microalgae (including a method of measuring the amount of lipid accumulation in the microalgae) according to an embodiment.

First, the culture of the microalga is started in the culture tank 50 (step S0).

Then, the fluid containing the microalgae is supplied to flow through the flow cell 40, and the flow cell 40 is irradiated with the excitation light from the excitation light source 10 (step S1).

Then, the fluorescence detector 102 detects the autofluorescence generated from the chloroplast of the microalga that has been irradiated with the excitation light in step S1. Furthermore, the scattered light detector 103 detects the scattered light caused by the microalga that has been irradiated with the excitation light in step S1. The size calculation unit 301 calculates the size of the microalga from the intensity of the scattered light having been detected. The quantitation unit 302 calculates the concentration of the microalgae from the volume of the fluid having passed through the flow cell during the unit time and the number of detection signals for the scattered light caused by the microalgae during the unit time (step S2).

Then, the ratio calculation unit 303 calculates (in step S3) the fluorescence density, namely the intensity of the autofluorescence from the chloroplast per unit size of the microalga, from the intensity of the autofluorescence from the chloroplast and the size of the microalga, both of which have been calculated in step S2.

Then, the lipid amount calculation unit 304 calculates the amount of lipid accumulation per number of the microalgae at the certain concentration from the fluorescence density that has been calculated in step S3. At that time, the previously obtained relation formula (see the above-described formula (1)) between the fluorescence density and the amount of lipid accumulation per number of the microalgae at the certain concentration can be used. On that occasion, preferably, the determination guide value (K) is calculated from the change over time of the cell size and the change over time of the fluorescence density in accordance with the above-described formula (2), and whether calculating the amount of lipid accumulation per number of the microalgae at the certain concentration based on the above-described relation formula is appropriate or not is determined in advance. Furthermore, the lipid amount calculation unit 304 calculates the amount of lipid accumulation per microalga from the amount of lipid accumulation per number of the microalgae at the certain concentration. Moreover, the lipid amount calculation unit 304 calculates (in step S4) the concentration of the lipids of the microalgae (namely, the total yield) from both the amount of lipid accumulation per microalga at the certain concentration (namely, the unit yield) and the concentration of the microalgae having been calculated in step S3.

Then, the evaluation unit 305 determines whether the unit yield calculated in step S4 exceeds the predetermined discriminant value (for example, the expected unit yield at the end of the culture) (step S5). If the unit yield does not exceed the expected unit yield at the end of the culture, the culture of the microalgae in the culture tank 50 is continued and steps S1 to S5 are repeatedly performed until the unit yield exceeds the expected unit yield while the culture solution is intermittently or continuously sampled.

If the unit yield exceeds the expected unit yield at the end of the culture in step S5, the evaluation unit 305 determines whether the total yield calculated in step S4 exceeds the predetermined discriminant value (for example, the expected total yield at the end of the culture) (step S6). If the total yield does not exceed the expected total yield at the end of the culture, the culture of the microalgae in the culture tank 50 is continued and steps S1 to S6 are repeatedly performed until the total yield exceeds the expected total yield while the culture solution is intermittently or continuously sampled.

If the total yield exceeds the expected total yield at the end of the culture in step S6, the evaluation unit 305 determines that it is time to end the culture of the microalgae. Then, in accordance with the above determination result of the evaluation unit 305, the output unit 501 issues, to the culture controller 60, the command to stop the culture. In accordance with the above command, the culture controller 60 ends the culture of the microalgae in the culture tank 50 (step S7).

In step S5 of the above-described method of controlling the culture of the microalgae, the evaluation unit 305 may further evaluate the state of the microalgae based on the unit yield and determine that it is time to adjust the culture conditions in the culture tank 50. For example, if the unit yield does not exceed the predetermined discriminant value (for example, the expected unit yield at the end of the culture), the evaluation unit 305 may determine that it is time to adjust the culture conditions of the microalgae. Then, in accordance with the above determination result of the evaluation unit 305, the output unit 501 issues, to the culture controller 60, the command to adjust the culture conditions. In accordance with the above command, the culture controller 60 adjusts the culture conditions in the culture tank 50. The culture conditions are adjusted in a manner of, for example, increasing the culture efficiency in the culture tank 50 and optimizing the culture conditions. Then, the culture of the microalgae in the culture tank 50 is continued under the adjusted culture conditions and steps S1 to S5 are repeatedly performed until the unit yield exceeds the predetermined discriminant value (for example, the expected unit yield) while the culture solution is intermittently or continuously sampled.

In step S6 of the above-described method of controlling the culture of the microalgae, the evaluation unit 305 may further evaluate the state of the microalgae based on the total yield and determine that it is time to adjust the culture conditions in the culture tank 50. For example, if the total yield does not exceed the predetermined discriminant value (for example, the expected total yield at the end of the culture), the evaluation unit 305 may determine that it is time to adjust the culture conditions of the microalgae. Then, in accordance with the above determination result of the evaluation unit 305, the output unit 501 issues, to the culture controller 60, the command to adjust the culture conditions. In accordance with the above command, the culture controller 60 adjusts the culture conditions in the culture tank 50. The culture conditions are adjusted in a manner of, for example, increasing the culture efficiency and optimizing the culture conditions. Then, the culture of the microalgae in the culture tank 50 is continued under the adjusted culture conditions and steps S1 to S6 are repeatedly performed until the total yield exceeds the predetermined discriminant value (for example, the expected total yield) while the culture solution is intermittently or continuously sampled.

The above-described method of controlling the culture of the microalgae according to this embodiment can provide similar advantageous effects to those described above in connection with the device of measuring the amount of lipid accumulation in the microalgae. In particular, since both the unit yield and the total yield can be accurately measured by the above-described method of controlling the culture of the microalgae, it is possible not only to check the culture efficiency and to determine the time to end the culture, but also to optimize the culture conditions.

### Experimental Example

Chlorella vulgaris Beijerinck was dispensed into a culture container, and preculture was performed under conditions as follows.

Culture medium: 200 mL TAP liquid medium (in culture container)

Temperature condition: 23° C.

Aeration condition: aerate a gas mixture of N₂:O₂:CO₂=77:20:3 at 20 mL/min

Light condition: irradiation with light from a fluorescence lamp (repeat irradiation for 10 hours and non-irradiation for 14 hours)

Culture time: 7 days

After the above preculture, the culture container was centrifuged to remove the culture medium in a supernatant. The culture medium was exchanged by adding 200 mL of a dN-TAP liquid medium into the culture container. Here, dN-TAP is a nitrogen-deficient TAP liquid medium that is obtained by removing ammonium chloride from the TAP liquid medium.

Then, the Chlorella after the exchange of the culture medium was subjected to main culture under similar conditions to those in the above-described preculture except for the culture medium and the culture time.

Then, BODIPY (registered trademark) 493/503, namely a lipid-labeling fluorescence dye with a peak wavelength of 503 nm, was prepared and a fluorescence reagent solution of 1 mg/mL was obtained by diluting the dye with ethanol. After the above main culture, a test tube was centrifuged for 3 min at 5000 rpm to remove the culture medium from a supernatant and to separate cell pellets. Then, a phosphate-buffered physiological saline solution was added to the separated cell pellets for resuspension. At that time, a resulting suspension was adjusted such that an optical density at a wavelength of 680 nm (namely, an OD₆₈₀ value) was held at 10 (0D₆₈₀=10).

Then, the fluorescence reagent solution obtained as described above was added at a concentration of 0.2% to the suspension including the cultured Chlorella, thus staining the Chlorella with BODIPY (registered trademark). Then, the fluorescence-stained suspension was put into a measurement cell, and the measurement cell was set in a fluorescence spectrophotometer (FP-8500 made by JASCO Corporation). Then, the measurement cell was irradiated with excitation light, and intensity of fluorescence generated from lipids of the microalgae due to fluorescence agents was measured by the fluorescence spectrophotometer. A xenon lamp was used as a light source to emit the excitation light, and light with a wavelength of 493 nm was obtained for the irradiation by using a spectrometer. The fluorescence with a wavelength of 508 nm was detected by using a spectrometer. A measurement value gives intensity of the fluorescence generated from the fluorescence agents in lipids per number of the microalgae at a certain concentration (OD₆₈₀=10). Here, the intensity of the fluorescence generated from the fluorescence agents in the lipids per number of the microalgae at the certain concentration reflects the amount of lipid accumulation per number of the microalga at the certain concentration.

On the other hand, a real-time microbe detector (IMD-W (registered trademark) made by Azbil Corporation) was prepared. IMD-W is a device capable of irradiating microbes flowing through a flow cell with excitation light and measuring scattered light and fluorescence generated from the microbes. The excitation light was a laser beam with a wavelength of 375 nm. A bandpass filter used to detect the fluorescence had a transmission wavelength range of 685±20 nm. Intensity of the scattered light caused by the cultured Chlorella and intensity of autofluorescence generated from a chloroplast were measured by IMD-W based on the principle of flowcytometry. During the measurement, a concentration of the culture solution was adjusted by adding an appropriate amount of deionized water (DW).

The main culture was performed for two weeks and, after the lapse of one day from the main culture, the culture solution was sampled per day or two days. The sampled culture solutions were each measured on the intensity of the fluorescence generated from the lipids due to the fluorescence staining, the intensity of the autofluorescence generated from the chloroplast, and the intensity of the scattered light.

Then, results of the above-described measurements were plotted on a graph with a horizontal axis representing the amount (X) of lipid accumulation per number of the microalgae at the certain concentration (equivalent to the fluorescence intensity due to the fluorescence agents in the lipids) and a vertical axis representing the fluorescence density (Y). Curve fitting was applied to the plotted points by using an approximate formula expressed by the above-described formula (1). Consequently, the coefficients a and b in the above-described formula (1) were determined. Under the above-described experimental conditions, a was 4179.5 and b was 0.879. Here, the mean square error (MSE) between the plotted points and the above-described approximate formula was 10.441, and the correlation coefficient R² was 0.862. From those values, it is seen that there is a strong correlation between the plotted points and the above-described approximate formula.

As mentioned above, the amount of lipid accumulation per number of the microalgae at the certain concentration (equivalent to the fluorescence intensity due to the fluorescence agents in the lipids) can be calculated from the fluorescence density by using the formula (1).

Although the embodiments of the present disclosure have been described in detail above, the present disclosure is not limited to the above-described embodiments and example and can be variously modified based on the technical concept of the present disclosure.

What is claimed is:

1. A device of measuring an amount of lipid accumulation in microalgae, the device comprising:
a flow cell through which a fluid containing the microalgae is supplied to flow;
an excitation light source irradiating the flow cell with excitation light;
a fluorescence detector detecting autofluorescence generated from a chloroplast of each of the microalgae that have been irradiated with the excitation light;
a scattered light detector detecting scattered light caused by each of the microalgae that have been irradiated with the excitation light; and
an arithmetic unit calculating:
a size of the microalga from intensity of the scattered light,
a fluorescence density corresponding to intensity of the autofluorescence generated from the chloroplast per unit size of the microalga based on both the intensity of the autofluorescence generated from the chloroplast and the size of the microalga, and
an amount of lipid accumulation per microalga from the fluorescence density.

2. The device of measuring the amount of lipid accumulation in the microalgae according to claim 1, wherein the arithmetic unit calculates:
an amount of lipid accumulation per number of the microalgae at a certain concentration from the fluorescence density, and
the amount of lipid accumulation per microalga from the amount of lipid accumulation per number of the microalgae at the certain concentration.

3. The device of measuring the amount of lipid accumulation in the microalgae according to claim 2, wherein the arithmetic unit further calculates:
a concentration of the microalgae from a volume of the fluid having passed through the flow cell during a unit time and a number of detection signals for the scattered light caused by the microalgae during the unit time, and
a concentration of lipids of the microalgae from the amount of lipid accumulation per microalga and the concentration of the microalgae.

4. The device of measuring the amount of lipid accumulation in the microalgae according to claim 3, wherein the arithmetic unit determines that it is time to end culture of the microalgae, when the amount of lipid accumulation per microalga and/or the concentration of the lipids of the microalgae exceeds a predetermined discriminant value.

5. The device of measuring the amount of lipid accumulation in the microalgae according to claim 4, further comprising an output unit issuing a command to stop the culture in a supply source for the fluid containing the microalgae in accordance with the determination that it is time to end the culture.

6. The device of measuring the amount of lipid accumulation in the microalgae according to claim 3, wherein the arithmetic unit evaluates a state of the microalgae based on the amount of lipid accumulation per microalga and/or the concentration of the lipids of the microalgae and determines that it is time to adjust culture conditions in a supply source for the fluid containing the microalgae.

7. The device of measuring the amount of lipid accumulation in the microalgae according to claim 6, further comprising an output unit issuing a command to adjust the culture conditions in the supply source for the fluid containing the microalgae in accordance with the determination that it is time to adjust the culture conditions.

8. The device of measuring the amount of lipid accumulation in the microalgae according to claim 1, further comprising a storage unit recording the intensity of the detected autofluorescence from the chloroplast and the intensity of the detected scattered light in chronological order.

9. The device of measuring the amount of lipid accumulation in the microalgae according to claim 8, wherein the storage unit further records the calculated amount of lipid accumulation per microalga and the calculated concentration of the lipids of the microalgae in chronological order.

10. The device of measuring the amount of lipid accumulation in the microalgae according to claim 1, further comprising a display unit displaying the calculated amount of lipid accumulation per microalga and/or the calculated concentration of the lipids of the microalgae.

11. A method of measuring an amount of lipid accumulation in microalgae, the method comprising:
supplying a fluid containing the microalgae to flow through a flow cell;
irradiating the flow cell with excitation light;
detecting autofluorescence generated from a chloroplast of each of the microalgae that have been irradiated with the excitation light;
detecting scattered light caused by each of the microalgae that have been irradiated with the excitation light;
calculating a size of the microalga from intensity of the scattered light;
calculating a fluorescence density corresponding to intensity of the autofluorescence generated from the chloroplast per unit size of the microalga based on both the intensity of the autofluorescence generated from the chloroplast and the size of the microalga; and
calculating an amount of lipid accumulation per microalga from the fluorescence density.

12. The method of measuring the amount of lipid accumulation in the microalgae according to claim 11, further comprising:
calculating an amount of lipid accumulation per number of the microalgae at a certain concentration from the fluorescence density; and
calculating the amount of lipid accumulation per microalga from the amount of lipid accumulation per number of the microalgae at the certain concentration.

13. The method of measuring the amount of lipid accumulation in the microalgae according to claim 11, further comprising:
calculating a concentration of the microalgae from a volume of the fluid having passed through the flow cell during a unit time and a number of detection signals for the scattered light caused by the microalgae during the unit time; and
calculating a concentration of lipids of the microalgae from the amount of lipid accumulation per microalga and the concentration of the microalgae.

14. A method of controlling culture of microalgae, the method comprising:
supplying a fluid containing the microalgae to flow through a flow cell;
irradiating the flow cell with excitation light;
detecting autofluorescence generated from a chloroplast of each of the microalgae that have been irradiated with the excitation light;
detecting scattered light caused by each of the microalgae that have been irradiated with the excitation light;
calculating a size of the microalga from intensity of the scattered light;
calculating a concentration of the microalgae from a volume of the fluid having passed through the flow cell during a unit time and a number of detection signals for the scattered light caused by the microalga during the unit time;
calculating a fluorescence density corresponding to intensity of the autofluorescence generated from the chloroplast per unit size of the microalga based on both the intensity of the autofluorescence generated from the chloroplast and the size of the microalga;
calculating an amount of lipid accumulation per number of the microalgae at a certain concentration from the fluorescence density;
calculating the amount of lipid accumulation per microalga from the amount of lipid accumulation per number of the microalgae at the certain concentration;
calculating a concentration of lipids of the microalgae from the amount of lipid accumulation per microalga and the concentration of the microalgae;
determining that it is time to end culture of the microalgae, when the amount of lipid accumulation per microalga and/or the concentration of the lipids of the microalgae exceeds a predetermined discriminant value; and
ending the culture of the microalgae in accordance with the determination that it is time to end the culture.

15. The method of controlling the culture of the microalgae according to claim 14, further comprising:
evaluating a state of the microalgae based on the amount of lipid accumulation per microalga and/or the concentration of the lipids of the microalgae and determining that it is time to adjust culture conditions in a supply source for the fluid containing the microalgae; and
adjusting the culture conditions in the supply source for the fluid containing the microalgae in accordance with the determination that it is time to adjust the culture conditions.
